(19) United States
(12) Patent Application Publication
(10) Pub. No.: US 2026/0166346 A1
(43) Pub. Date: Jun. 18, 2026

(54) ULTRASOUND HANDPIECE

(71) Applicant: VIOL CO., LTD., Seongnam-si (KR)

(72) Inventors: Yong Seok PARK, Seoul (KR); Sanghyeon HWANG, Hanam-si (KR)

(21) Appl. No.: 18/711,972

(22) PCT Filed: Jul. 31, 2023
(86) PCT No.: PCT/KR2023/011167

§ 371 Date: May 21, 2024

(30) Foreign Application Priority Data
Aug. 22, 2022 (KR) 10-2022-0104925

(51) Int. Cl. A61N 7/00

(52) U.S. Cl. CPC A61N 7/00; A61N 2007/0008; A61N 2007/0034

(57) ABSTRACT

The present disclosure relates to an ultrasonic handpiece including a body; an ultrasonic generator movably provided on one side of the body; a position adjustment unit provided on the body and configured to adjust a position of the ultrasonic generator so that a focusing position of ultrasonic waves of the ultrasonic generator is adjusted; a position detection unit configured to detect a movement position of the ultrasonic generator; and a control unit configured to control an output of the ultrasonic generator according to the position of the ultrasonic generator detected by the position detection unit.

As a result, an output of ultrasonic waves may be controlled according to the focusing depth of the ultrasonic waves.

### TECHNICAL FIELD

The present disclosure relates to an ultrasonic handpiece, and more particularly, to an ultrasonic handpiece capable of detecting a position of an ultrasonic generator.

### BACKGROUND ART

Ultrasonic handpieces include ultrasonic generators that may irradiate ultrasonic waves to the human body.

Some of these ultrasonic handpieces are used in procedures to focus ultrasonic waves on the inside of the human skin to deteriorate or decompose subcutaneous fat.

Others of these ultrasonic handpieces perform a so-called ‘lifting’ skin care procedure to focus relatively weak ultrasonic waves on the inside of the human skin, generate thermal coagulation points in the area, and coagulate (shrink) the skin tissue.

However, when performing skin care procedures using such conventional ultrasonic handpieces, there is a problem in that it is difficult to focus the ultrasonic waves on a desired position because skin thicknesses of operators are different.

In consideration of such a problem, an ultrasonic handpiece has been designed to adjust a position of an ultrasonic generator so that a focusing position of ultrasonic waves may be adjusted from a point where the ultrasonic handpiece contacts the skin.

However, in the ultrasonic handpiece with such a position adjustment function, because ultrasonic outputs are consistently output regardless of a focusing depth of the ultrasonic waves irradiated to the human body, when a procedure is performed at a procedure depth relatively close to the epidermis, the human body is exposed to more output than necessary, and thus, there is a problem that injuries such as burns occur in the human body.

Considering such a problem, some of the conventional ultrasonic handpieces are limited to a relatively low output with respect to the entire procedure depth to prevent injuries (burns) due to application of a relatively high output compared to a relatively shallow procedure depth, and thus, there is a problem that the procedure effect of the entire procedure depth is relatively insufficient.

### PRIOR ART DOCUMENT

### Patent Document

(Patent Document 1) KR 101578490 B1

(Patent Document 2) KR 102256560 B1

(Patent Document 3) KR 1020210108801 A

### DISCLOSURE

### Technical Problem

Therefore, the present disclosure attempts to provide an ultrasonic handpiece in which an output of ultrasonic waves may be controlled according to a focusing depth of the ultrasonic waves.

In addition, the present disclosure also attempts to provide an ultrasonic handpiece capable of preventing injuries to the human body due to an excessive output of ultrasonic waves.

In addition, the present disclosure also attempts to provide an ultrasonic handpiece capable of outputting different ultrasonic waves at different procedure depths, thereby improving the procedure effect.

### Technical Solution

The ultrasonic handpiece according to the present disclosure for solving the problems described above may output different ultrasonic waves according to procedure depths.

Specifically, the ultrasonic handpiece may detect a position of a movable ultrasonic generator and control an output of the ultrasonic generator based on a position detection result of the ultrasonic generator, thereby suppressing the occurrence of injuries due to an excessive output.

According to an embodiment of the present disclosure, an ultrasonic handpiece includes a body; an ultrasonic generator movably provided on one side of the body; a position adjustment unit provided on the body and configured to adjust a position of the ultrasonic generator so that a focusing position of ultrasonic waves of the ultrasonic generator is adjusted; a position detection unit configured to detect a movement position of the ultrasonic generator; and a control unit configured to control an output of the ultrasonic generator according to the position of the ultrasonic generator detected by the position detection unit.

As a result, the output of ultrasonic waves may be appropriately controlled to correspond to the focusing depth of the ultrasonic waves.

In addition, an excessive output of the ultrasonic waves is suppressed compared to the focusing depth of the ultrasonic waves, and thus, the occurrence of injuries (burns) to the human body due to the excessive output may be suppressed.

In addition, when the focusing depth of the ultrasonic waves is relatively deep, the output of the ultrasonic waves is relatively increased, and thus, the ultrasonic procedure effect may be improved.

According to an embodiment of the present disclosure, the position adjustment unit may include a dial rotatable relative to the body; and an action rod provided inside the body, and having one side in contact with the dial to enable relative movement, and the other side connected to the ultrasonic generator and moved in an axial direction to correspond to a rotation of the dial.

As a result, the action rod is moved in the axial direction to correspond to the rotation of the dial, and thus, the position of the ultrasonic generator may be accurately adjusted.

According to an embodiment of the present disclosure, a groove in a spiral shape may be provided inside of the dial, and a protrusion that moves along the groove may be provided on the action rod.

As a result, during the rotation of the dial, the protrusion is pressed by the groove in the axial direction, and thus, the action rod may be moved in the axial direction.

According to an embodiment of the present disclosure, the action rod has an axial direction period disposed in the axial direction and a radial direction period disposed in a radial direction with respect to the axial direction period,
the radial direction period has a bent end that is bent in the axial direction, and
the protrusion is provided on the bent end and elastically contacts the groove.

As a result, the occurrence of a gap between the dial and the action rod may be suppressed.

In addition, the occurrence of a slip in the dial and the action rod may be suppressed.

According to an embodiment of the present disclosure, the protrusion may have a curved shape that is convexly outward, and
a plurality of expansion portions expanded to correspond to a shape of the protrusion and spaced apart from each other may be provided in the groove.

As a result, when the protrusion contacts the expansion portion, a contact area increases, and thus, a coupling state of the protrusion and the expansion portion may be stably maintained.

According to such a configuration, the occurrence of the gap in the action rod is suppressed and the occurrence of gap in the ultrasonic generator is suppressed, and thus, the focusing depth of the ultrasonic waves of the ultrasonic generator may be stably maintained.

According to an embodiment of the present disclosure, a pin may be provided on one side of the protrusion, and
a pin guide portion may be provided on one side of the groove to correspond to a movement trajectory of the pin.

As a result, the coupling state of the dial and the action rod may be more stably maintained.

In addition, during the rotation of the dial, the movement of the action rod in the axial direction may be more accurately performed.

According to an embodiment of the present disclosure, the body may include a main body; and a cartridge detachably coupled to the main body,
the ultrasonic generator may be provided inside the cartridge.

As a result, the ultrasonic generator may be easily replaced.

According to an embodiment of the present disclosure, the action rod may include a front rod connected to the ultrasonic generator; a rear rod in contact with the dial; and a middle rod having one end connected to the front rod and the other end connected to the rear rod.

As a result, a length of the action rod may be reduced, and thus, manufacturing and assembly may be easier.

In addition, when replacing the cartridge, the length of the discarded action rod may be reduced.

In addition, the occurrence of deformation due to the long length of the action rod may be suppressed.

According to an embodiment of the present disclosure, the front rod and the middle rod may respectively include magnetic coupling portions that are magnetically coupled to each other.

As a result, when the cartridge and the body are coupled to each other, the front rod and the middle rod may be stably coupled.

Here, the magnetic coupling portion may include a permanent magnet and a permanent magnet accommodation portion in which the permanent magnet is accommodated.

The permanent magnet accommodation portion may be provided in a contact region between the front rod and the middle rod, and the permanent magnet may be accommodated and coupled to the inside of the permanent magnet accommodation portion.

According to an embodiment of the present disclosure, the middle rod and the rear rod may include engaging coupling portions that are engaged and coupled to be constrained in the axial direction and circumferential direction.

As a result, the middle rod and the rear rod are restrained in the axial direction and circumferential direction, respectively, and thus, the motion (movement) of the rear rod in the axial direction that occurs during the rotation manipulation of the dial may be accurately transmitted to the middle rod.

According to an embodiment of the present disclosure, the position detection unit may include
a light emitting portion configured to irradiate light;
a light receiving portion configured to detect the light from the light emitting portion; and
a blocking portion configured to interoperate with an action rod and disposed to be movable in an axial direction between the light emitting portion and the light receiving portion to selectively block the light from the light emitting portion.

As a result, the position of the ultrasonic generator according to the movement of the action rod may be accurately detected.

According to an embodiment of the present disclosure, the light emitting portion may include a first light emitting portion and a second light emitting portion spaced apart in the axial direction,
the first light emitting portion may be disposed closer to the ultrasonic generator than the second light emitting portion,
the blocking portion may have an axial length capable of simultaneously blocking the first light emitting portion and the second light emitting portion, and
the dial and the action rod may be configured to be fixed to a first position where the blocking portion blocks the second light emitting portion, a second position where the blocking portion simultaneously blocks the first light emitting portion and the second light emitting portion, and a third position where the blocking portion blocks the first light emitting portion.

As a result, it is possible to accurately detect that the ultrasonic generator is moved to any one of the first position, the second position, and the third position and present.

Here, the first position may be configured to have a focusing depth of the ultrasonic waves of about 1.5 mm,
the second position may be configured to have a focusing depth of the ultrasonic waves of about 3.0 mm, and
the third position may be configured to have a focusing depth of the ultrasonic waves of about 4.5 mm.

According to an embodiment of the present disclosure, the position detection unit may include
a first position detection unit including the light emitting portion and the blocking portion and provided inside the cartridge to detect a movement of the action rod; and
a second position detection unit including the light emitting portion and the blocking portion and provided inside the main body to detect the movement of the action rod.

As a result, the movement (position) of the action rod inside the cartridge and the movement (position) of the action rod inside the body may be detected, and thus, when the cartridge and the body are coupled to each other, a coupling state of the front rod and the middle rod may be accurately determined (detected).

According to an embodiment of the present disclosure, the control unit may control driving of the ultrasonic generator to be stopped when detection results of the first position detection unit and the second position detection unit are different.

As a result, an excessive or insufficient output of the ultrasonic waves compared to the focusing depth of the ultrasonic waves of the ultrasonic generator may be suppressed.

According to an embodiment of the present disclosure, the ultrasonic handpiece may further include a display unit provided on the body to display preset information on the outside of the body, and
the control unit may control the display unit to display an error on the outside of the body when the detection results of the first position detection unit and the second position detection unit are different.

As a result, a user may easily determine an incomplete coupling state of the front rod and the middle rod and take an early measure.

According to such a configuration, the possibility that the ultrasonic generator is to operate at a position different from a position manipulated by the dial may be significantly reduced.

### Advantageous Effects

As described above, according to an embodiment of the present disclosure, by providing the position detection unit that detects the position of the ultrasonic generator and the control unit that controls the output of the ultrasonic generator based on a detection result of the position detection unit, the output of ultrasonic waves may be appropriately controlled to correspond to the focusing depth ultrasonic waves.

In addition, by suppressing the excessive output of the ultrasonic waves compared to the focusing depth of the ultrasonic waves, the occurrence of injuries (burns) to the human body due to the excessive output may be suppressed.

In addition, when the focusing depth of the ultrasonic waves is relatively deep, by relatively increasing the output of the ultrasonic waves, the ultrasonic procedure effect may be improved.

In addition, a separate power source for adjusting the position of the ultrasonic generator is not included, and thus, the weight and volume of the handpiece are reduced, and convenience of use is increased, thereby reducing the fatigue of the operator.

In addition, a separate power source for adjusting the position of the ultrasonic generator is not included, and thus, reduction in the manufacturing costs and simplification of production procedures may be expected.

In addition, by providing the dial that may be rotatable with respect to the body and the action rod that is moved in the axial direction in correspondence to the rotation of the dial, the position of the ultrasonic generator may be accurately adjusted.

In addition, the spiral groove is provided inside the dial, and the protrusion that moves along the groove is provided in the action rod, so that the protrusion is pressed by the groove, and thus, the ultrasonic generator may be accurately moved in the axial direction.

In addition, the action rod has the axial direction period disposed in the axial direction and the radial direction period disposed in the radial direction with respect to the axial direction period, the radial direction period has the bent end bent in the axial direction, and the protrusion is provided on the bent end and elastically contacts the groove, and thus, the occurrence of the gap and the slip between the dial and the action rod may be suppressed.

In addition, the protrusion has the outwardly convex curved shape, and the groove includes the plurality of expansion portions that expands to correspond to the shape of the protrusion and are spaced apart from each other, and thus, a coupling state of the protrusion and the expansion portions may be stably maintained.

In addition, the pin is provided on one side of the protrusion, and the pin guide portion is provided on one side of the groove to correspond to the movement trajectory of the pin, and thus, the coupling state of the dial and the action rod may be more stably maintained.

In addition, the body includes the main body and the cartridge detachably coupled to the main body, and the ultrasonic generator is provided inside the cartridge, and thus, the ultrasonic generator may be easily replaced.

In addition, the action rod includes the front rod connected to the ultrasonic generator, the rear rod in contact with the dial, and the middle rod having one end connected to the front rod and the other end connected to the rear rod, and thus, the length of the action rod may be reduced, making manufacturing and assembly easier. In addition, when replacing the cartridge, the length of the discarded action rod may be reduced.

In addition, the position detection unit includes the light emitting portion that irradiates light, the light receiving portion that detects the light from the light emitting portion, and the blocking portion that is disposed to be movable in the axial direction between the light emitting portion and the light receiving portion and selectively blocks the light from the light emitting portion, and thus, the position of the ultrasonic generator according to the movement of the action rod may be accurately detected.

In addition, the light emitting portion includes a first light emitting portion and a second light emitting portion spaced apart from each other in the axial direction, the first light emitting portion is disposed farther from the ultrasonic generator than the second light emitting portion, the blocking portion is configured to have an axial length capable of blocking the first light emitting portion and the second light emitting portion at the same time, and the dial and the action rod are fixedly located at a first position where the blocking portion blocks the first light emitting portion, a second position where the blocking portion blocks the first light emitting portion and the second light emitting portion at the same time, and a third position where the blocking portion blocks the second light emitting portion, and thus, it is possible to accurately detect that the ultrasonic generator is moved to any one of the first position, the second position, and the third position and present.

In addition, the position detection unit includes a first position detection unit provided in the cartridge and a second position detection unit provided inside the body, thereby accurately determining (detecting) the coupling state of the front rod and the middle rod when the cartridge and the body are coupled to each other.

In addition, when detection results of the first position detection unit and the second position detection unit are different, the control unit controls the display unit to display an error on the outside of the body so that a user may easily identify an incomplete coupling state of the front rod and the middle rod and take an early measure.

### DESCRIPTION OF THE DRAWINGS

FIG. 1 is a perspective view of an ultrasonic handpiece according to an embodiment of the present disclosure;

FIG. 2 is a cross-sectional view of the ultrasonic handpiece of FIG. 1;

FIG. 3 is an exploded perspective view of the ultrasonic handpiece of FIG. 1;

FIG. 4 is an exploded perspective view of a cartridge of FIG. 3;

FIG. 5 is an exploded perspective view of a main body of FIG. 3;

FIG. 6 is an exploded perspective view of a position adjustment unit of FIG. 3;

FIG. 7 is a perspective view of a rear rod and a dial of FIG. 6;

FIG. 8 is a perspective view of the rear rod of FIG. 6;

FIG. 9 is a perspective view of a front rod and a middle rod of FIG. 2;

FIG. 10 is a perspective view of the middle rod and the rear rod of FIG. 2;

FIG. 11 is a perspective view of a position detection unit of FIG. 2;

FIG. 12 is a diagram showing a first position of the position detection unit of FIG. 11;

FIG. 13 is a diagram showing a second position of the position detection unit of FIG. 11;

FIG. 14 is a diagram showing a third position of the position detection unit of FIG. 11;

FIG. 15 is a control block diagram of the ultrasonic handpiece of FIG. 1;

FIG. 16 is a cross-sectional view of an ultrasonic handpiece according to another embodiment of the present disclosure;

FIG. 17 is a diagram showing a first position detection unit and a second position detection unit of FIG. 16; and

FIG. 18 is a control block diagram of the ultrasonic handpiece of FIG. 16.

### MODE FOR INVENTION

Hereinafter, embodiments disclosed in the present specification will be described in detail with reference to the attached drawings. In the present specification, the same or similar reference numerals are assigned to the same or similar components even in different embodiments, and the descriptions thereof are replaced with the first descriptions. Singular expressions used in the present specification include plural expressions unless the context clearly dictates otherwise. In addition, in describing the embodiments disclosed in the present specification, when it is determined that detailed descriptions of related known technologies may obscure the gist of the embodiments disclosed in the present specification, the detailed descriptions thereof will be omitted. In addition, it should be noted that the attached drawings are only for easy understanding of the embodiments disclosed in the present specification, and should not be construed as limiting the technical idea disclosed in the present specification by the attached drawings.

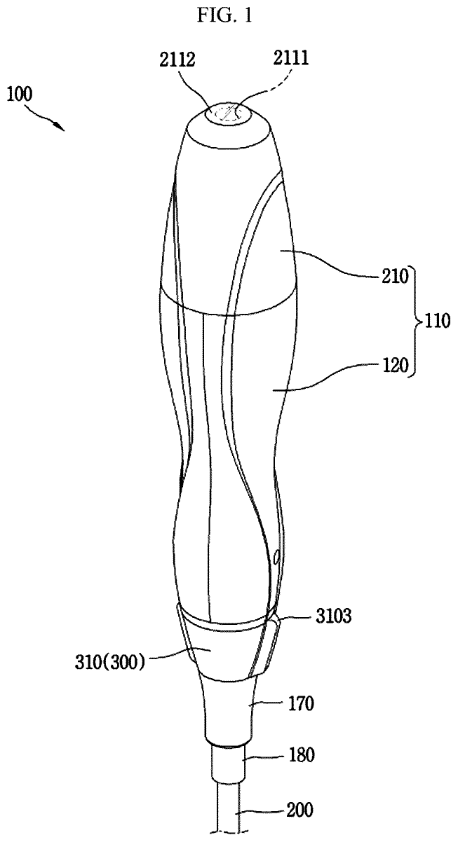

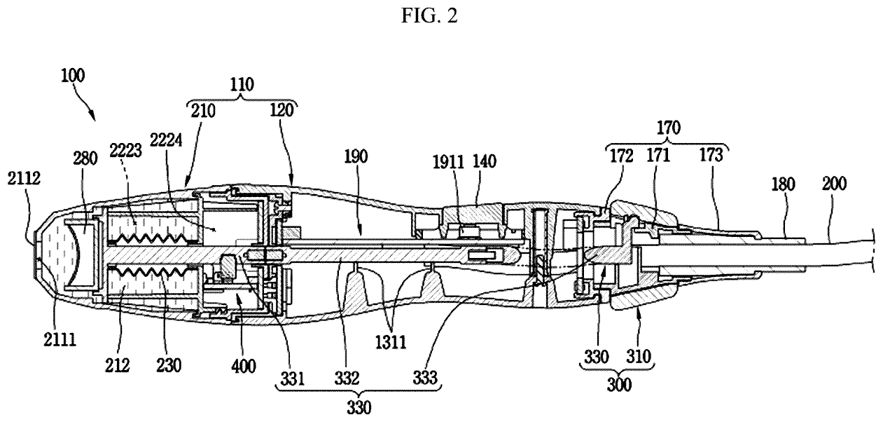

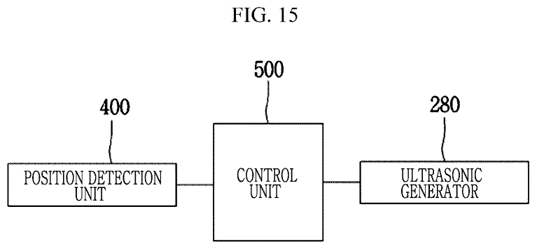

FIG. 1 is a perspective view of an ultrasonic handpiece according to an embodiment of the present disclosure. FIG. 2 is a cross-sectional view of the ultrasonic handpiece of FIG. 1. As shown in FIGS. 1 and 2, an ultrasonic handpiece 100 of the present embodiment includes a body 110, an ultrasonic generator 280, a position adjustment unit 300, a position detection unit 400, and a control unit 500 (see FIG. 15).

The body 110 may be implemented, for example, in a circular rod shape.

The body 110 may be configured to have, for example, an outer diameter that gradually changes in an axial direction.

In the present embodiment, the axial direction means a direction parallel to a longitudinal direction of the body 110 or a direction parallel to a movement direction of the ultrasonic generator 280, which will be described below, moved by the position adjustment unit 300.

Specifically, both ends of the body 110 have a relatively small outer diameter and are implemented in a shape that the outer diameter gradually increases from the both ends toward the center in the axial direction and decreases again in a central region.

The ultrasonic generator 280 is provided inside the body 110.

The ultrasonic generator 280 is provided at one end (upper end in the drawing) of the inside of the body 110.

The ultrasonic generator 280 is configured to, for example, irradiate ultrasonic waves at an acute angle so as to focus the ultrasonic waves.

In the present embodiment, the ultrasonic generator 280 may be implemented as, for example, an ultrasonic transducer.

In the present embodiment, the ultrasonic generator 280 is configured to focus the ultrasonic waves on a point spaced by a preset distance from one end (upper end in the drawing) of the body 110.

The ultrasonic generator 280 is configured to be movable with respect to the body 110 so as to adjust a focusing depth of the ultrasonic waves irradiated from the ultrasonic generator 280.

The ultrasonic generator 280 is configured to be movable in the axial direction inside the body 110.

The position adjustment unit 300 includes, for example, a dial 310 and an action rod 330.

The dial 310 is configured to be rotatable with respect to the body 110.

The action rod 330 is provided inside the body 110 and has one side in contact with the dial 310 to enable relative movement and the other side connected to the ultrasonic generator 280.

As a result, the action rod 330 may be moved in the axial direction during the rotation of the dial 310.

The action rod 330 includes, for example, an axial direction period 330a disposed in the axial direction and a radial direction period 330b disposed in a radial direction with respect to the axial direction period 330a.

The action rod 330 may be formed of, for example, a plurality of parts in the axial direction.

Specifically, the action rod 330 may include, for example, a front rod 331 connected to the ultrasonic generator 280, a rear rod 333 in contact with the dial 310, and a middle rod 332 having one end connected to the front rod 331 and the other side connected to the rear rod 333.

In the present embodiment, the case in which the action rod 330 is divided into three parts in the axial direction is illustrated, but this is only an example and is not limited thereto.

When the action rod 330 is configured as a single portion, the front rod 331 and the middle rod 332 may be formed integrally with the axial direction period 330a of the rear rod 333.

In addition, when the action rod 330 is configured as two parts, the middle rod 332 may be formed integrally with the axial direction period 330a of the rear rod 333.

Meanwhile, a through hole 2111 is provided at one end (upper end in the drawing) of the body 110 to allow the ultrasonic waves irradiated from the ultrasonic generator 280 to pass therethrough.

A liquid medium 212 may be filled around the ultrasonic generator 280.

The liquid medium 212 may be implemented as, for example, water.

The through hole 2111 may be sealed.

As a result, leakage of the liquid medium 212 to the outside through the through hole 2111 may be suppressed.

Specifically, for example, the through hole 2111 may be blocked by a film 2112 attached to an end of the body 110.

For example, the film 2112 may be provided (attached) to the outside or inside of the end of the body 110 where the through hole 2111 is formed.

A driving button 140 for driving the ultrasonic generator 280 is provided in the body 110.

The driving button 140 is configured such that one side (outer surface) thereof is exposed to the outside of the body 110.

As a result, the driving button 140 may be manipulated from the outside of the body 110.

The driving button 140 may be configured to enable, for example, a pressing manipulation.

A printed circuit board (PCB) 190 is provided inside the body 110.

A switch 1911 is provided in the PCB 190 so as to be in contact with the driving button 140.

The switch 1911 is configured to allow an operation signal to be input during the pressing manipulation of the driving button 140.

One end of a cable 200 withdrawn to the outside of the body 110 is electrically connected to the PCB 190.

The cable 200 passes through the other end (lower end in the drawing) of the body 110 to be withdrawn to the outside.

In the present embodiment, the end of the body 110 in which the ultrasonic generator 280 is provided may be referred to as a front end, and the end of the body 110 from which the cable 200 is withdrawn may be referred to as a rear end.

The other end of the cable 200 is connected to a control console (not shown) provided outside the body 110.

As a result, the PCB 190 may receive power from and communicate with the control console through the cable 200.

Meanwhile, the body 110 may include, for example, a main body 120 and a cartridge 210 that is detachably coupled to the main body 120.

In the present embodiment, the main body 120 constitutes a middle and rear region of the body 110, and the cartridge 210 constitutes a front region of the body 110, but this is an example, and is not limited thereto.

The position detection unit 400 includes, for example, a light emitting portion 401, a light receiving portion 402, and a blocking portion 403.

For example, the light emitting portion 401 and the light receiving portion 402 may be arranged horizontally (vertical direction) in a movement direction of the action rod 330 (ultrasonic generator 280).

For example, the blocking portion 403 may be moved in the axial direction in conjunction with the action rod 330.

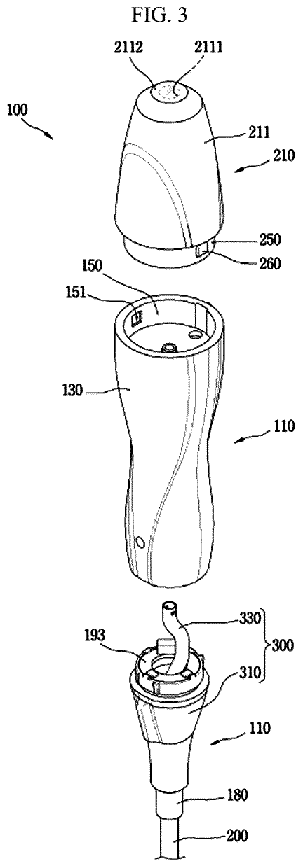

FIG. 3 is an exploded perspective view of the ultrasonic handpiece 100 of FIG. 1. As shown in FIG. 3, the body 110 includes the main body 120 and the cartridge 210.

A cartridge coupling portion 150 to which the cartridge 210 is detachably coupled is formed in the main body 120.

An insertion portion 250 is provided in the cartridge 210 so as to be inserted into the main body 120.

For example, the insertion portion 250 has a reduced outer diameter compared to the maximum outer diameter of the cartridge 210.

A coupling protrusion 260 that protrudes and withdraws in the radial direction is provided in the cartridge 210. Specifically, a spring may be provided behind the coupling protrusion 260 to apply an elastic force so that the coupling protrusion 260 protrudes outward in the radial direction.

The coupling protrusion 260 may be implemented, for example, in a rectangular cross-sectional shape.

The coupling protrusion 260 may be implemented, for example, in plural.

A coupling protrusion accommodation portion 151 is provided in the main body 120 (cartridge coupling portion 150) so as to accommodate and be coupled to the coupling protrusion 260.

The coupling protrusion accommodation portion 151 is implemented in a shape corresponding to the shape of the coupling protrusion 260.

In the present embodiment, because the coupling protrusion 260 has the rectangular cross-sectional shape, the coupling protrusion accommodation portion 151 is implemented in the rectangular cross-sectional shape.

Meanwhile, the position adjustment unit 300 is provided in the main body 120.

As described above, the position adjustment unit 300 includes the dial 310 and the action rod 330.

The position adjustment unit 300 is, for example, provided in a rear region of the main body 120.

Specifically, the main body 120 includes, for example, a housing 130 and a dial support member 170 that is coupled to the housing 130 and rotatably supports the dial 310.

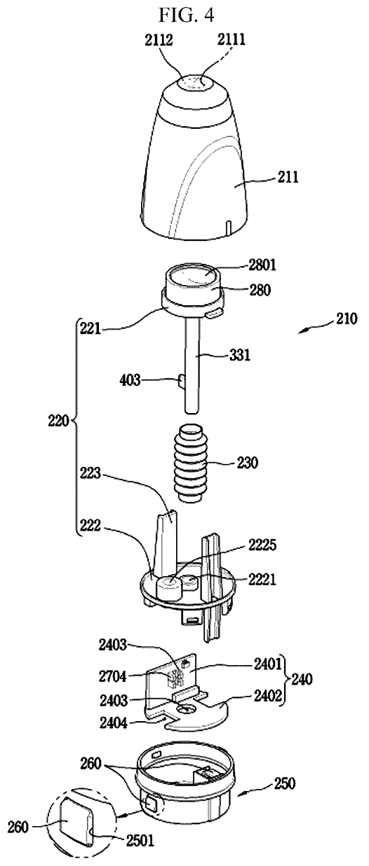

FIG. 4 is an exploded perspective view of the cartridge 210 of FIG. 3. As shown in FIG. 4, the cartridge 210 includes a cartridge body 211, the ultrasonic generator 280, an ultrasonic generator support portion 220, and the insertion portion 250.

The cartridge body 211 forms an accommodation space therein.

The cartridge body 211 is implemented in a substantially cylindrical shape.

The through hole 2111 is formed in one end (upper end in the drawing) of cartridge body 211.

The ultrasonic generator 280 is implemented to irradiate ultrasonic waves to converge to one side.

The ultrasonic generator 280 has a cylindrical shape.

An irradiation surface 2801 in the shape of a concave lens is provided on one side (upper side in the drawing) of the ultrasonic generator 280.

As a result, the ultrasonic waves may be irradiated at an acute angle and converge (focus) on one point.

The ultrasonic generator 280 is supported by the ultrasonic generator support portion 220.

The ultrasonic generator support portion 220 includes, for example, an accommodation portion 221 in which one side of the ultrasonic generator 280 is accommodated and coupled, a partition portion 222 that is spaced apart from the accommodation portion 221 in the axial direction and partitions an inner space of the cartridge body 211, and a connection portion 223 that connects the partition portion 222 to the accommodation portion 221.

The partition portion 222 is implemented in a disk shape.

A first accommodation space 2223 in which the liquid medium 212 is accommodated is formed in one inner side (left side of FIG. 2) formed by the cartridge body 211 and the partition portion 222, and a second accommodation space 2224 in which a cartridge PCB 240 is accommodated is formed in the other side (right side of FIG. 2).

A sealing member may be provided between the partition portion 222 and the inner surface of the cartridge body 211.

As a result, leakage of the liquid medium 212 through a gap between the cartridge body 211 and the partition portion 222 may be prevented.

One end of the action rod 330 is connected to one side of the accommodation portion 221.

Specifically, the front rod 331 is connected to the accommodation portion 221.

The front rod 331 penetrates and is coupled to the partition portion 222.

A through hole 2225 through which the front rod 331 may be accommodated is provided in the partition portion 222.

An injection port 2221 through which the liquid medium 212 is injected is formed in the partition portion 222.

An injection port stopper that blocks the injection port 2221 is provided in the injection port 2221.

An expandable corrugated pipe 230 is provided around the front rod 331.

One end of the corrugated pipe 230 is coupled to the accommodation portion 221 and the other end is coupled to the partition portion 222.

As a result, contact between the front rod 331 and the liquid medium 212 may be suppressed.

The insertion portion 250 is provided in one side (lower side in the drawing) of the partition portion 222 in the axial direction.

The insertion portion 250 is implemented, for example, in a cylindrical shape.

In the present embodiment, one side (upper side in the drawing) of the insertion portion 250 is inserted into the inside of the cartridge body 211, and the other side (lower side in the drawing) thereof is inserted into the main body 120.

The connection portion 223 may extend to the other side (lower side in the drawing) of the partition portion 222.

The end (lower end in the drawing) of the connection portion 223 may be connected to the insertion portion 250.

The coupling protrusion 260 is coupled to the insertion portion 250.

The end of the coupling protrusion 260 protrudes from the outside of the insertion portion 250.

The coupling protrusion 260 is implemented as, for example, a pair.

A coupling protrusion hole 2501 is formed through the insertion portion 250 so that the pair of coupling protrusions 260 may be moved in the radial direction.

The position detection unit 400 is provided in the cartridge PCB 240.

Specifically, the cartridge PCB 240 includes, for example, a first PCB 2401 disposed in the axial direction and a second PCB 2402 disposed in the radial direction.

The first PCB 2401 is implemented, for example, in the form of a rectangular plate.

The second PCB 2402 is implemented, for example, in a disk shape.

A cutting portion 2404 cut into a rectangular shape is provided in the second PCB so as to accommodate the coupling protrusion 260.

A through hole 2403 through which the action rod 330 (front rod 331) may be accommodated to enable relative movement is provided in the second PCB 2402.

The position detection unit 400 includes the light emitting portion 401 and the light receiving portion 402 disposed to be spaced apart horizontally in the movement direction of the action rod 330, and the blocking portion 403 disposed to be in conjunction with the action rod 330.

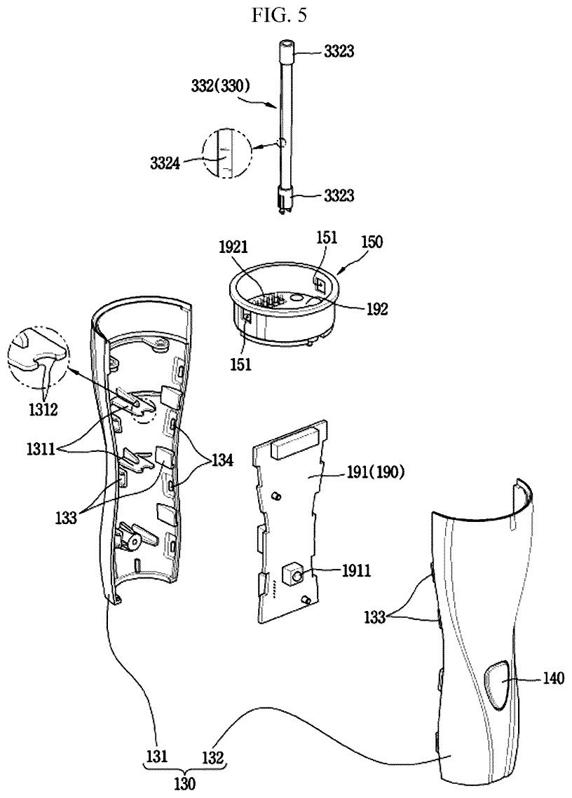

FIG. 5 is an exploded perspective view of the main body of FIG. 3. As shown in FIG. 5, the main body 120 includes the housing 130 and the PCB 190.

The housing 130 includes, for example, a first housing 131 and a second housing 132 that are coupled to each other in face-to-face contact.

The first housing 131 and the second housing 132 are configured to have, for example, a semicircular cross-sectional shape.

A hook 133 and a hook coupling portion 134 are provided in a mutual contact region of the first housing 131 and the second housing 132, respectively.

The hooks 133 and the hook coupling portions 134 are formed alternately with each other in the axial direction.

Specifically, the hook 133 of the first housing 131 is coupled to the hook coupling portion 134 of the second housing 132.

The hook 133 of the second housing 132 is coupled to the hook coupling portion 134 of the first housing 131.

As a result, the first housing 131 and the second housing 134 may be firmly coupled to each other.

The driving button 140 is provided in the housing 130 so as to enable pressing manipulation.

In the present embodiment, the driving button 140 is provided in the second housing 132, but is not limited thereto.

The cartridge coupling portion 150 is provided on one side (upper side in the drawing) of the housing 130.

The cartridge coupling portion 150 is implemented, for example, in a cylindrical shape.

Each of coupling protrusion accommodation portions 151 is formed through the cartridge coupling portion 150 to accommodate the coupling protrusion 260 of the cartridge 210.

The PCB 190 is provided inside the housing 130.

The PCB 190 inside the housing 130 includes, for example, a first PCB 191 on a plate, a second PCB 192 provided on one side (front) of the first PCB 191, and a third PCB provided on the other side (rear) of the first PCB 191.

Here, the first PCB 191, the second PCB 192, and the third PCB 193 are electrically connected to each other.

The first PCB 191 is implemented, for example, in a long plate shape.

The first PCB 191 is disposed in the axial direction.

The switch 1911 in contact with the driving button 140 to input a manipulation signal is provided in the first PCB 191.

The second PCB 192 is implemented in a disk shape.

The second PCB 192 is disposed in front of the first PCB 191.

The second PCB 192 is disposed, for example, inside the cartridge coupling portion 150.

The third PCB 193 is disposed, for example, in the rear region (lower end in the drawing) of the housing 130 (see FIGS. 2 and 3).

The third PCB 193 is disposed behind the first PCB 191.

The second PCB 192 is configured to be electrically connected to the second PCB of the cartridge PCB 240.

A plurality of pogo pins 1921 are provided in the second PCB 192.

The plurality of pogo pins 1921 are electrically connected to the cartridge PCB (second PCB 2402).

The action rod 330 is provided inside the housing 130.

Specifically, the middle rod 332 of the action rod 330 is provided inside the housing 130.

The middle rod 332 is implemented in a rod shape.

Expansion ends 3323 having expanded diameters are respectively provided in both ends of the middle rod 332.

A guide groove 3324 that is recessed in the radial direction and extends in the axial direction is provided between the expansion ends 3323 of the middle rod 332.

A plurality of guide grooves 3324 are formed in an outer surface of the middle rod to be spaced apart in a circumferential direction.

In the present embodiment, the guide grooves 3324 are configured as two and are arranged 180 degrees apart in the circumferential direction.

An action rod support portion 1311 that supports the middle rod 332 to be movable in the axial direction is provided inside the housing 130.

The action rod support portion 1311 is, for example, disposed horizontally in the axial direction inside the first housing 131.

The action rod support portion 1311 is implemented, for example, in plural spaced apart in the axial direction.

Semicircular cutting portions are formed at ends of the action rod support portions 1311 so as to partially accommodate the action rod 330 (middle rod 332).

Guide protrusions 1312 that are inserted into the guide grooves 3324 are respectively provided on ends of the cutting portions.

The guide protrusions 1312 are implemented, for example, as a pair respectively disposed on both sides with the middle rod 332 therebetween.

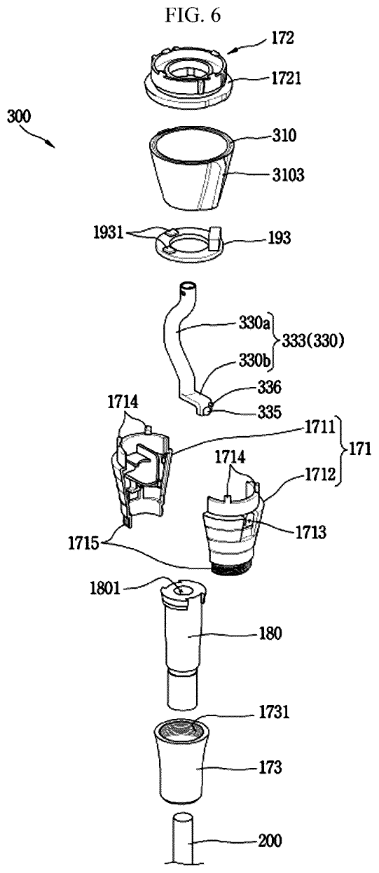

FIG. 6 is an exploded perspective view of the position adjustment unit 300 of FIG. 3. As shown in FIG. 6, the position adjustment unit 300 includes the dial 310 and the operating rod 330.

The dial 310 is implemented in a cylindrical shape.

Specifically, the dial 310 may be implemented in a truncated cone cross-sectional shape.

A handle 3103 that protrudes in the radial direction and extends in the axial direction may be provided in an outer surface of the dial 310.

The handle 3103 may be implemented, for example, as a plurality of handles spaced apart in the circumferential direction.

In the present embodiment, the handle 3103 is implemented as two handles spaced apart at 180 degrees in the circumferential direction, but this is only an example and is not limited thereto.

The dial 310 may be rotatably supported on the dial support member 170.

The dial support member 170 may include, for example, a first support member 171 disposed inside the dial 310, a second support member 172 provided on one side (lupper side in the drawing) of the first support member 171 in the axial direction, and a third support member 173 provided on the other side (lower side in the drawing) of the first support member 171.

The first support member 171 includes, for example, a first member 1711 and a second member 1712 that are coupled to face each other in the radial direction.

The first member 1711 and the second member 1712 are each configured to have a semicircular cross-sectional shape.

The first member 1711 and the second member 1712 form a circular cross-section when coupled to each other.

An accommodation hole 1713 is formed through one side of the first support member 171 so as to accommodate a part (a protrusion 335 to be described below) of the action rod 330 (rear rod 333).

For example, the accommodation hole 1713 is configured to have a long length in the axial direction so that the protrusion 335 of the action rod 330 may be moved in the axial direction.

A coupling member 1714 is provided in the first support member 171 so as to be coupled to the second support member 172.

The coupling member 1714 of the first support member 171 may be implemented as, for example, a plurality of hooks.

The plurality of hooks may be, for example, inserted into the second support member 172 in the axial direction and then spread outward in the radial direction to be constrained in the radial direction and in the axial direction, respectively.

As a result, after the first support member 171 and the second support member 172 are coupled to each other, the occurrence of gap in the axial direction and gap in the radial direction between the first support member 171 and the second support member 172 may be suppressed.

The second support member 172 is implemented, for example, in a cylindrical shape.

One side (upper side in the drawing) of the second support member 172 is inserted into and coupled to the inside of the housing 130.

A stopper 1721 that contacts one end (upper end in the drawing) of the dial 310 and suppresses a movement of the dial 310 in the axial direction is provided in the second support member 172.

The stopper 1721 is implemented in a disk shape to expand outward in the radial direction.

The third support member 173 is implemented, for example, in a cylindrical shape.

The third support member 173 is configured to be, for example, screw-coupled with the first support member 171.

A male thread portion 1715 is provided on one side (lower side in the drawing) of the first support member 171.

A female thread portion 1731 into which the male thread portion 1715 may be screwed is provided on one side (upper side in the drawing) of the third support member 173.

The third support member 173 may be configured to have different outer diameters at both ends.

The third support member 173 is configured to have a larger outer diameter at an upper end than at a lower end in the drawing.

The third support member 173 is configured to have one end (upper end in the drawing) in surface contact with one end (lower end in the drawing) of the dial 310.

As a result, separation of the dial 310 in the axial direction may be suppressed.

The end of the third support member 173 on the first support member 171 functions as a stopper.

A cable guide 180 in which the cable 200 is accommodated is provided inside the dial support member 170.

A cable accommodation hole 1801 penetrating in the axial direction is provided in the cable guide 180.

The third PCB 193 of the PCB 190 described above is disposed on one side (upper side in the drawing) of the second support member 172.

The third PCB 193 has a disk shape.

Specifically, the third PCB 193 may be implemented in an annular shape having an inner diameter and an outer diameter of a preset size.

For example, a plurality of LEDs 1931 may be provided in the third PCB 193.

The plurality of LEDs 1931 may be implemented to, for example, emit light in different colors.

Specifically, for example, the plurality of LEDs 1931 may include a green LED and a red LED.

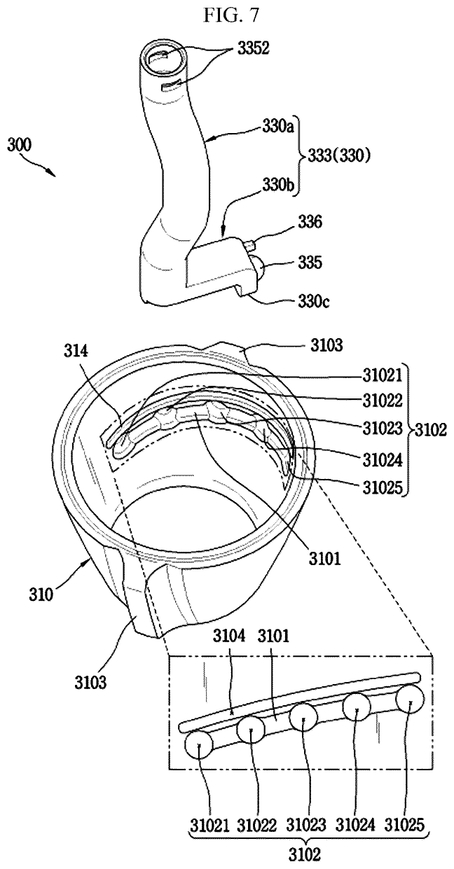

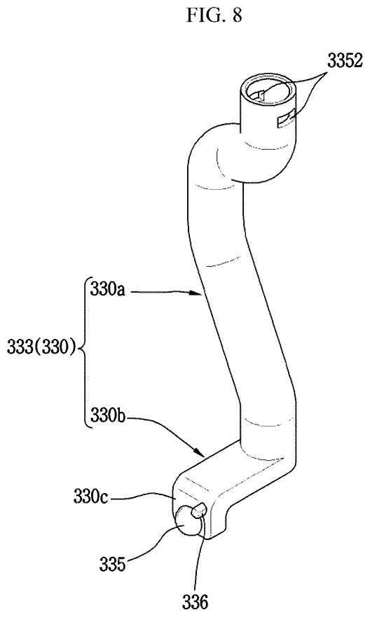

FIG. 7 is a perspective view of the rear rod 333 and dial 310 of FIG. 6, and FIG. 8 is a perspective view of the rear rod 333 of FIG. 6. As shown in FIGS. 7 and 8, the action rod 330 includes the axial direction period 330a and the radial direction period 330b, as described above.

As described above, the action rod 330 includes the front rod 331, the middle rod 332, and the rear rod 333 that are spaced apart in the axial direction.

The rear rod 333 includes, for example, the axial direction period 330a and the radial direction period 330b.

The axial direction period 330a of the rear rod 333 has a bent cylindrical shape.

Both ends of the rear rod 333 are arranged to be spaced apart from each other in the axial direction.

The central region of the rear rod 333 has a shape that is convexly bent outward.

The radial direction period 330b is provided on one side (lower side in the drawing) of the axial direction period 330a of the rear rod 333.

The radial direction period 330b has, for example, a rectangular cross-sectional shape.

The radial direction period 330b is configured to have, for example, a greater width in the radial direction than a width in the axial direction.

A bent end 330c bent to be disposed in the axial direction is provided in the end of the radial direction period 330b.

The protrusion 335 is provided in the bent end portion 330c.

The protrusion 335 has a curved shape that is convex outward.

For example, the protrusion 335 may be implemented in a hemispherical shape that is convex outward.

A pin 336 is provided at the bent end 330c.

The pin 336 protrudes outward from an outer surface of the bent end portion 330c.

The pin 336 is provided on one side (upper side in the drawing) of the protrusion in the axial direction.

A groove 3101 in a spiral shape is provided inside the dial 310.

The protrusion 335 of the action rod 330 is coupled to the groove 3101 to enable relative movement.

An expansion portion 3102 that expands to correspond to the shape of the protrusion 335 is formed in the groove 3101.

The protrusion 335 may be accommodated in surface contact with the inside of the expansion portion 3102.

As a result, the protrusion 335 may be stably accommodated and fixed (temporarily fixed).

According to such a configuration, when the protrusion 335 is accommodated in the expansion portion 3102, the occurrence of movement (gap) of the protrusion 335 may be suppressed, which may suppress the occurrence of gap in the ultrasonic generator 280, and thus, a focusing depth of ultrasonic waves may be stably maintained.

The expansion portion 3102 is configured as, for example, a plurality of expansion portions spaced apart from each other at a preset distance (angle) in the circumferential direction of the dial 310.

For example, the expansion portions 3102 may be spaced apart, for example, at an angle of 30 degrees.

The expansion portion 3102 may include, for example, a first expansion portion 31021 to a fifth expansion portion 31025 spaced apart in the circumferential direction.

Here, the first expansion portion 31021 is formed at a position closest to the ultrasonic generator 280.

The fifth expansion portion 31025 is formed at a position farthest from the ultrasonic generator 280.

The protrusions 335 may be sequentially moved from the first expansion portion 31021 to the fifth expansion portion 31025.

In the present embodiment, a case in which the expansion portions 3102 are implemented as five is illustrated, but the embodiment is not limited thereto.

An angle between the two expansion portions 3102 disposed at both ends in the circumferential direction, that is, the first expansion portion 31021 and the fifth expansion portion 31025, may be 120 degrees.

In the present embodiment, the groove 3101 of the dial 310 is configured such that the action rod 330 may be moved by a preset length when the dial 310 is rotated at a preset angle.

For example, in the present embodiment, when the dial 310 is rotated 30 degrees, the action rod 330 may be configured to be moved by 1.5 mm in the axial direction.

Specifically, for example, in the present embodiment, when the protrusion 335 is located in the first expansion portion 31021, the focusing depth of the ultrasonic waves irradiated from the ultrasonic generator 280 is 0, that is, focused on the epidermis,

When the protrusion 335 is located in the second expansion portion 31022, the focusing depth of the ultrasonic waves emitted from the ultrasonic generator 280 may be 1.5 mm inward from the epidermis.

When the protrusion 335 is located in the third expansion portion 31023, the focusing depth of the ultrasonic waves emitted from the ultrasonic generator 280 may be 3.0 mm inward from the epidermis.

In addition, when the protrusion 335 is located in the fourth expansion portion 31024, the focusing depth of the ultrasonic waves irradiated from the ultrasonic generator 280 may be 4.5 mm inward from the epidermis.

When the protrusion 335 is located in the fifth expansion portion 31025, the focusing depth of the ultrasonic waves emitted from the ultrasonic generator 280 may be 6.0 mm inward from the epidermis.

A pin guide portion 3104 guiding the pin 336 is provided on the inner surface of the dial 310.

The pin guide portion 3104 may be implemented, for example, to be recessed from the inner surface of the dial 310.

The pin guide portion 3104 may be formed parallel to the groove 3101.

As a result, the occurrence of gap between the action rod 330 and the dial 310 may be suppressed.

According to such a configuration, the occurrence of gap in the ultrasonic generator 280 is suppressed, and thus, the focusing depth of ultrasonic waves may be stably maintained.

Meanwhile, the front rod 331 and the middle rod 332 may be configured, for example, to be magnetically coupled to each other.

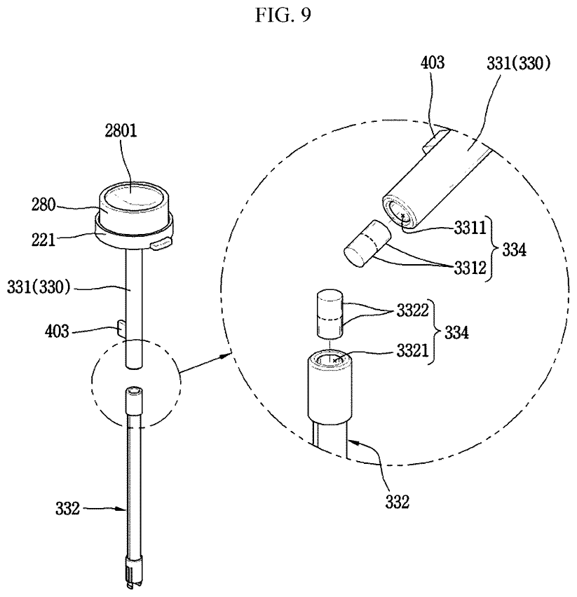

FIG. 9 is a perspective view of the front rod 331 and middle rod 332 of FIG. 2. As shown in FIG. 9, magnetic coupling portions 334 are formed in a mutual contact region between the front rod 331 and the middle rod 332.

The magnetic coupling portion 334 includes, for example, a permanent magnet 3322 and a permanent magnet accommodation portion 3321 in which the permanent magnet 3322 is accommodated.

The permanent magnets 3322 respectively provided on the front rod 331 and the middle rod 332 have different magnetic poles (N pole and S pole) so that surfaces in contact with each other may be attracted to each other.

As a result, when the cartridge 210 and the body 110 are coupled to each other, the front rod 331 and the middle rod 332 may be accurately and stably coupled to each other.

The permanent magnet 3322 may be implemented, for example, in a disk shape.

The permanent magnets 3322 may be implemented, for example, in plural.

In the present embodiment, a case where the permanent magnets 3322 is each configured as two is illustrated, but the embodiment is not limited thereto.

The permanent magnet accommodation portions 3311 and 3321 may be provided, for example, on the front rod 331 and the middle rod 332, respectively.

The permanent magnet accommodation portions 3311 and 3321 are respectively recessed to be formed in the axial direction at one end (lower end in the drawing) of the front rod 331 and one end (upper end in the drawing) of the middle rod 332.

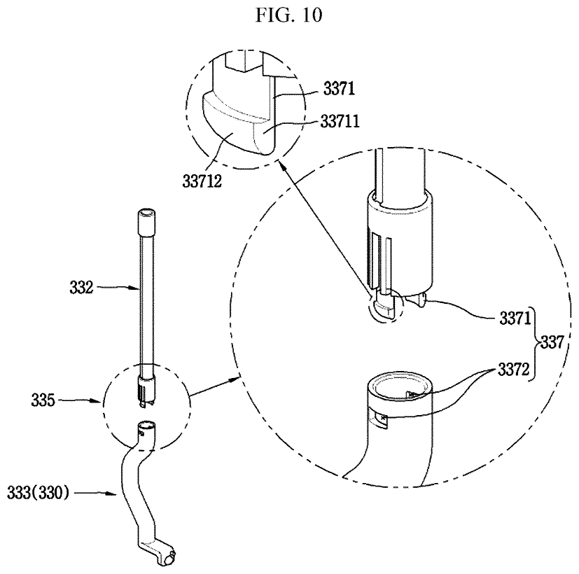

FIG. 10 is a perspective view of the middle rod 332 and the rear rod 333 of FIG. 2. As shown in FIG. 10, the middle rod 332 and the rear rod 333 include engaging coupling portions 337 that are engaged and coupled to be constrained in the axial direction and the circumferential direction, respectively.

As a result, the middle rod 332 and the rear rod 333 are restrained in the axial direction and the circumferential direction, respectively, and thus, a motion (movement) of the rear rod 333 in the axial direction that occurs during the rotation of the dial 310 may be accurately transmitted to the middle rod 332.

The engaging coupling portion 337 includes, for example, a hook 3371 and a hook accommodation portion 3372 where the hook 3371 is accommodated and coupled.

In the present embodiment, the hook 3371 is provided on the middle rod 332, and the hook accommodation portion 3372 is provided on the rear rod 333.

The hook 3371 may be implemented, for example, as a pair arranged to face each other.

The hook 3371 is supported by, for example, a hook support portion 33711 that protrudes in the axial direction from one end (lower end in the drawing) of the middle rod 332.

Because the hook support portion 33711 has a cantilever shape, the hook support portion 33711 may be elastically deformed by its own elasticity.

As a result, the hook 3371 is capable of, for example, elastic displacement in the radial direction of the middle rod 332.

A guide inclined surface 33712 is provided on an outer surface of the hook 3371.

The guide inclined surface 33712 is formed to be inclined inward farther away from the middle rod 332 in the axial direction.

The hook accommodation portion 3372 is formed through, for example, one end (upper end in the drawing) of the rear rod 333.

According to such a configuration, when a front end (lower end in the drawing) of the hook 3371 is pressed against one side (upper side in the drawing) of the rear rod 333, the hook 3371 is inserted into the rear rod 333 while being elastically deformed in a direction approaching each other (inner direction).

When the hooks 3371 respectively reach the hook accommodation portions 3372, the hooks 3371 are opened to an initial position (outside) by their own elastic force and are respectively accommodated and coupled to the inside of the corresponding hook accommodation portions 3372.

As a result, the hooks 3371 are restrained in the axial direction, the radial direction, and the circumferential direction, respectively.

As a result, the middle rod 332 and the action rod 330 are integrated so that gaps in the axial direction, the radial direction, and the circumferential direction may be respectively suppressed.

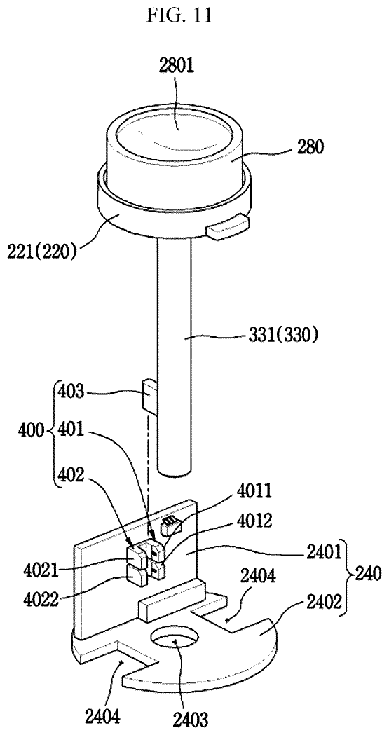

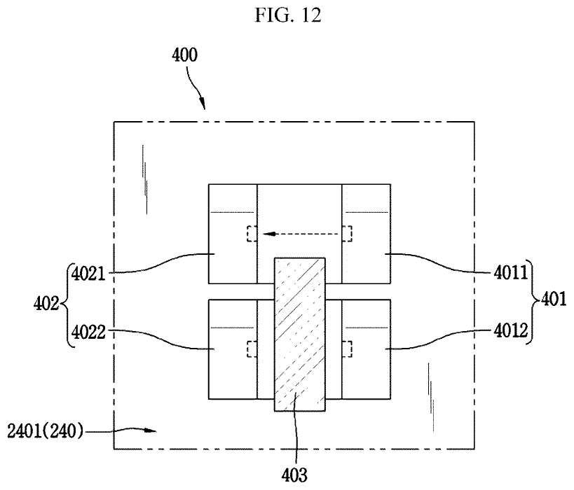

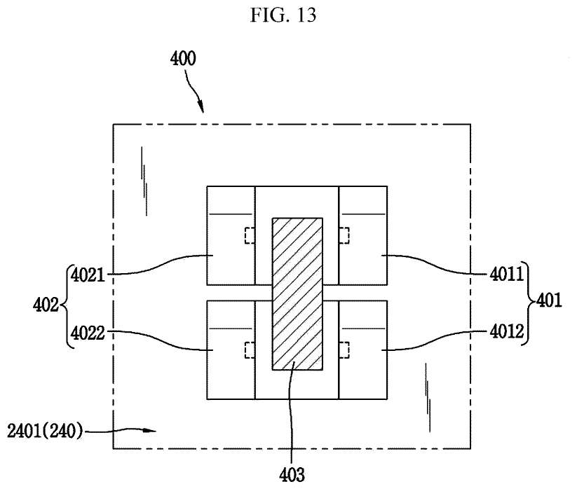

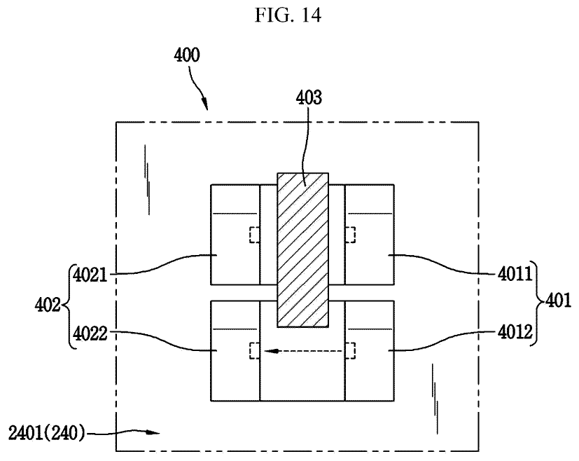

FIG. 11 is a perspective view of a position detection unit 400 of FIG. 2. FIG. 12 is a diagram showing a first position of the position detection unit 400 of FIG. 11. FIG. 13 is a diagram showing a second position of the position detection unit 400 of FIG. 11. FIG. 14 is a diagram showing a third position of the position detection unit 400 of FIG. 11.

As shown in FIGS. 11 to 14, the position detection unit 400 includes the light emitting portion 401, the light receiving portion 402, and the blocking portion 403.

The light emitting portion 401 is configured to emit light.

The light receiving portion 402 is configured to detect the light emitted from the light emitting portion 401.

The light emitting portion 401 and the light receiving portion 402 are arranged to face each other.

The light emitting portion 401 and the light receiving portion 402 are provided in the cartridge PCB 240.

The light emitting portion 401 and the light receiving portion 402 are arranged in a pair.

In the present embodiment, the light emitting portion 401 and the light receiving portion 402 are arranged horizontally in a movement direction of the action rod 330.

Here, the light emitting portion 401 and the light receiving portion 402 are spaced apart from each other at a distance greater than a thickness of the blocking portion 403.

That is, the light emitting portion 401 and the light receiving portion 402 may be transmission photosensors.

The light emitting portion 401 is configured, for example, in plural to be spaced apart in the movement direction of the action rod 330.

The light emitting portion 401 includes a first light emitting portion 4011 and a second light emitting portion 4012 spaced apart in the axial direction.

The first light emitting portion 4011 is disposed close to the ultrasonic generator 280.

The second light emitting portion 4012 is disposed at a position relatively far from the ultrasonic generator 280.

The light receiving portion 402 includes a first light receiving portion 4021 and a second light receiving portion 4022 spaced apart in the axial direction.

The blocking portion 403 is provided between the light emitting portion 401 and the light receiving portion 402.

The blocking portion 403 is configured to, for example, selectively block the light from the light emitting portion 401.

The blocking portion 403 is configured to, for example, interoperate with the action rod 330.

Specifically, the blocking portion 403 is coupled to the action rod 330.

The blocking portion 403 may be configured to, for example, protrude in the radial direction and be coupled to one side of the action rod 330 (front rod 331).

The blocking portion 403 is configured to, for example, have a length (width) in the axial direction capable of simultaneously blocking the first light emitting portion 4011 and the second light emitting portion 4012.

The blocking portion 403 may be implemented, for example, in the shape of a rectangular plate.

Specifically, the blocking portion 403 may be moved between the first position for blocking the second light emitting portion 4012, the second position for simultaneously blocking the first light emitting portion 4011 and the second light emitting portion 4012, and the third position for blocking the first light emitting portion 4011.

The protrusion 335 of the position adjustment unit 300 may be located on any one of the plurality of expansion portions 3102 formed inside the dial 310.

Specifically, for example, when the protrusion 335 is located on the first expansion portion 31021 formed closest to the ultrasonic generator 280 among the five expansion portions 3102 described above, the light from the first light emitting portion 4011 and the second light emitting portion 4012 may all be detected by the first light receiving portion 4021 and the second light receiving portion 4022.

When the protrusion 335 moves to the second expansion portion 3102 along the groove 3101, the ultrasonic generator 280 is located at the first position.

At the first position, the ultrasonic waves generated by the ultrasonic generator 280 may be focused on a point spaced 1.5 mm inward from the epidermis.

When the protrusion 335 is moved to the third expansion portion 3102, the ultrasonic generator 280 is located at the second position.

At the second position, the ultrasonic waves generated by the ultrasonic generator 280 may be focused on a point spaced 3.0 mm inward from the epidermis.

When the protrusion 335 is moved to the fourth expansion portion 3102, the ultrasonic generator 280 is moved to the third position.

At the third position, the ultrasonic waves generated by the ultrasonic generator 280 may be focused on a point spaced 4.5 mm inward from the epidermis.

As shown in FIG. 12, the first light emitting portion 4011 may operate when the blocking portion 403 is at the first position.

At the first position, the light from the first light emitting portion 4011 may be detected by the first light receiving portion 4021.

As shown in FIG. 13, the first light emitting portion 4011 and the second light emitting portion 4012 may operate at the second position.

At the second position, neither the light from the first light emitting portion 4011 nor the second light emitting portion 4012 is detected.

As shown in FIG. 14, the second light emitting portion 4012 may operate at the third position.

At the third position, the light from the second light emitting portion 4012 may be detected by the second light receiving portion 4022.

FIG. 15 is a control block diagram of the ultrasonic handpiece 100 of FIG. 1. As shown in FIG. 15, the ultrasonic handpiece 100 of the present embodiment includes the control unit 500 that has a control program and is implemented as a microprocessor.

The control unit 500 is configured to control an output of the ultrasonic generator 280 according to a position of the ultrasonic generator 280 detected by the position detection unit 400.

The position detection unit 400 that detects the position of the ultrasonic generator 280 is communicatively connected to the control unit 500.

The control unit 500 may be provided in, for example, the PCB 190, but is not limited thereto.

For another example, the control unit 500 may be provided in a device (e.g., control console) that is electrically connected to the ultrasonic handpiece 100.

The ultrasonic generator 280 is controllably connected to the control unit 500.

For example, the control unit 500 may control the ultrasonic waves output from the ultrasonic generator 280 to be limited to a preset first output when the ultrasonic generator 280 is at the first position.

Here, the first output may be set to an output smaller than the maximum output of the ultrasonic generator 280.

As a result, when the ultrasonic waves are focused on a point close to the epidermis, it is possible to suppress the occurrence of injury to the human body due to excessive ultrasonic waves output from the ultrasonic generator 280.

The control unit 500 may control, for example, the output of the ultrasonic generator 280 to output the maximum output at the second position and the third position.

Accordingly, at the second position and the third position, the ultrasonic generator 280 may output the ultrasonic waves of relatively high intensity, thereby improving the ultrasonic procedure effect.

In addition, in another embodiment, the control unit 500 may control, for example, the output of the ultrasonic generator 280 to be limited to a second output at the second position.

Here, the second output may be set to a value greater than the first output and less than the maximum output.

According to such a configuration, by applying different outputs according to a focusing depth of the ultrasound waves spaced from the epidermis to the inside, it is possible to prevent injury to the human body and improve the ultrasonic procedure effect.

In the present embodiment, when the protrusion 335 is located in the first expansion portion 31021 or the fifth expansion portion 31025, the control unit 500 may control driving of the ultrasonic generator 280 to be stopped.

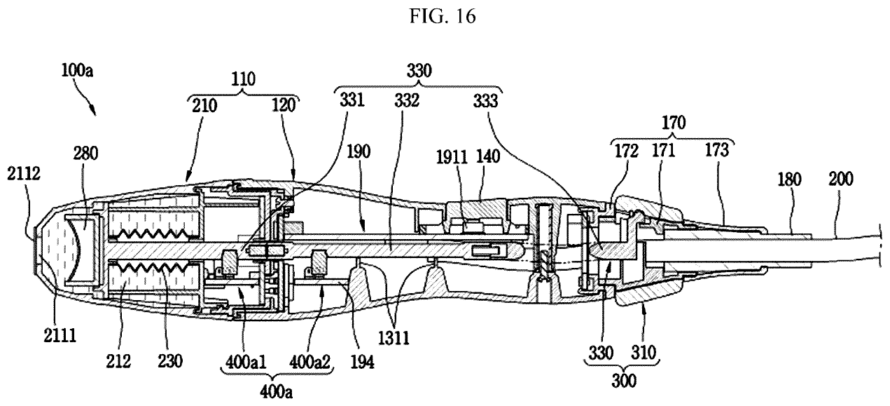

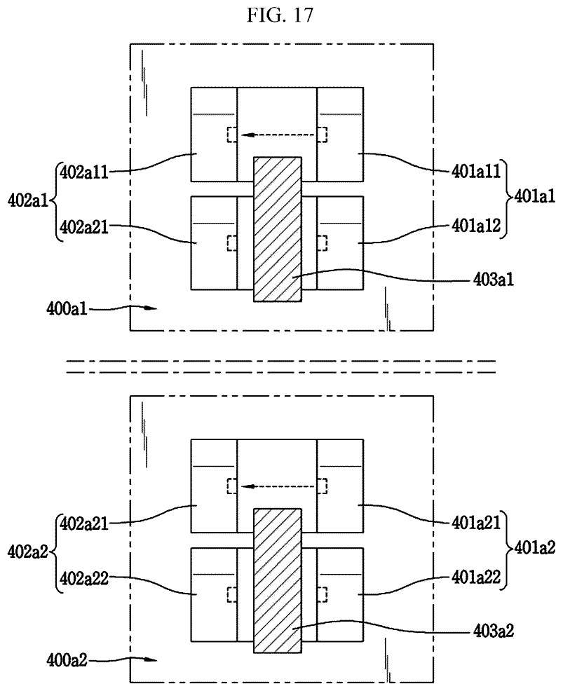

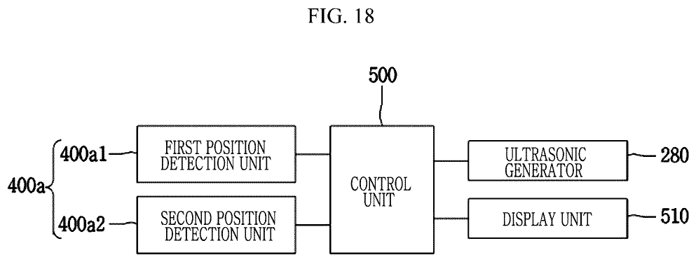

FIG. 16 is a cross-sectional view of an ultrasonic handpiece 100a according to another embodiment of the present disclosure. FIG. 17 is a diagram showing a first position detection unit 400a1 and a second position detection unit 400a2 of FIG. 16. FIG. 18 is a control block diagram of the ultrasonic handpiece 100a of FIG. 16. As shown in FIG. 16, the ultrasonic handpiece 100a of the present embodiment includes the body 110, the ultrasonic generator 280, the position adjustment unit 300, a position detection unit 400a, and the control unit 500.

The body 110 has a circular rod shape.

The ultrasonic generator 280 is provided inside the body 110.

The ultrasonic generator 280 is installed to be movable in the axial direction.

The position adjustment unit 300 that adjusts a position of the ultrasonic generator 280 is provided in the body 110.

The position adjustment unit 300 includes the dial 310 and the action rod 330.

The dial 310 and the action rod 330 are configured to enable the ultrasonic generator 280 to move between a first position, a second position, and a third position as described above.

The dial 310 is provided to be rotatable with respect to the body 110.

The action rod 330 is provided inside the body 110 and has one side in contact with the dial 310 to enable relative movement and the other side connected to the ultrasonic generator 280.

The action rod 330 may include the front rod 331 connected to the ultrasonic generator 280, the rear rod 333 in contact with the dial 310, and the middle rod 332 having one end connected to the front rod 331 and the other end connected to the rear rod 333.

The action rod 330 includes the axial direction period 330a disposed in the axial direction and the radial direction period 330b disposed in the radial direction with respect to the axial direction period 330a.

The body 110 may, for example, include the main body 120 and the cartridge 210 that is detachably coupled to the main body 120.

The driving button 140 that drives the ultrasonic generator 280 is provided in the main body 120.

The PCB 190 is provided inside the main body 120.

The switch 1911 in contact with the driving button 140 to input a manipulation signal is provided in the driving button 140.

The PCB 190 is connected to a control console (not shown) provided outside the body 110 by the cable 200 to enable power supply and communication.

The cartridge 210 is configured to be detachable from the main body 120.

Meanwhile, the position detection unit 400a includes, for example, the first position detection unit 400a1 disposed inside the cartridge 210 and the second position detection unit 400a2 provided inside the main body 120.

The first position detection unit 400a1 is configured to detect, for example, a movement of the action rod 330 (front rod 331) inside the cartridge 210.

The second position detection unit 400a2 is configured to detect, for example, a movement of the action rod 330 (middle rod 332) inside the main body 120.

Specifically, as shown in FIG. 17, the first position detection unit 400a1 includes a light emitting portion 401a1 and a light receiving portion 402a1 disposed on one side of the action rod 330 horizontally in the axial direction, and a blocking portion 403a1 disposed to selectively block light from the light emitting portion 401a1.

The light emitting portion 401a1 includes a first light emitting portion 401a11 and a second light emitting portion 401a12 spaced apart in the axial direction.

The light receiving portion 402a1 includes a first light receiving portion 402a11 and a second light receiving portion 402a12 spaced apart in the axial direction.

The blocking portion 403a1 of the first position detection unit 400a1 is provided on the front rod 331 so as to interoperate with the front rod 331.

The light emitting portion 401a1 and the light receiving portion 402a1 of the first position detection unit 400a1 are provided in the cartridge PCB 240 provided inside the cartridge 210.

The second position detection unit 400a2 includes a light emitting portion 401a2 and a light receiving portion 402a2 disposed inside the body 110 (main body 120) horizontally in the axial direction, and a blocking portion 403a2 disposed to selectively block light from the light emitting portion 401a2.

The light emitting portion 401a2 of the second position detection unit 400a2 includes a first light emitting portion 401a21 and a second light emitting portion 401a22 spaced apart in the axial direction.

The light receiving portion 402a2 of the second position detection unit 400a2 includes a first light receiving portion 402a21 and a second light receiving portion 402a22 spaced apart in the axial direction.

The blocking portion 403a2 of the second position detection unit 400a2 is provided on the middle rod 332 so as to interoperate with the middle rod 332.

The light emitting portion 401a2 and the light receiving portion 402a2 of the second position detection unit 400a2 are provided in the fourth PCB 190 provided inside the main body 120.

Meanwhile, as shown in FIG. 18, the ultrasonic handpiece 100a of the present embodiment includes the control unit 500 that has a control program and is implemented as a microprocessor.

The control unit 500 is configured to control an output of the ultrasonic generator 280 based on detection results of the first position detection unit 400a1 and the second position detection unit 400a2.

The first position detection unit 400a1 and the second position detection unit 400a2 are each connected to the control unit 500 to enable communication.

The ultrasonic generator 280 is controllably connected to the control unit 500.

For example, when the ultrasonic generator 280 is located at the first position, the control unit 500 may be configured to limit a first output smaller than the maximum output of the ultrasonic generator 280.

As a result, the occurrence of injury (burn) to the human body due to excessive focusing of the ultrasonic waves at a point relatively close to the epidermis may be suppressed.

The control unit 500 may control the ultrasonic generator 280 to be output at the maximum output when the ultrasonic generator 280 is disposed at each of the second and third positions.

As a result, the ultrasonic procedure effect may be improved at the second position and third position.

A display unit 510 is controllably connected to the control unit 500.

The display unit 510 is configured to, for example, previously display preset information on the outside of the body 110.

In the present embodiment, the display unit 510 includes, for example, the plurality of LEDs 1931.

As a result, the control unit 500 may control the plurality of LEDs 1931 to display different visual information on the outside.

The control unit 500 may compare detection results of the first position detection unit 400a1 and the second position detection unit 400a2, and, when position information of the ultrasonic generator 280 are different, control output of the ultrasonic generator 280 to be stopped.

The control unit 500 may compare the detection results of the first position detection unit 400a1 and the second position detection unit 400a2, and, when position information of the ultrasonic generator 280 are different, control the display unit 510 (plural of LEDs 1931) to display errors on the outside. For example, the control unit 500 may control the LED 1931 of a preset color to light or flicker among the plurality of LEDs 1931 so that errors may be displayed on the outside.

As a result, the ultrasonic generator 280 may operate at a position different from the preset position, thereby previously preventing injury (burn) to the human body from occurring.

In addition, defective coupling between the cartridge 210 and the main body 120 may be identified at an early stage, and thus, appropriate measures may be taken at an early stage.

In the above, specific embodiments of the present disclosure have been shown and described. However, because the present disclosure may be implemented in various forms without departing from its spirit or essential characteristics, the embodiments described above should not be limited by the specific details for carrying out the invention.

In addition, even if embodiments are not individually listed in the detailed description described above, the embodiments should be interpreted broadly within the scope of the technical idea defined in the appended claims. In addition, all changes and modifications that fall within the technical scope of the claims and their equivalents shall be encompassed by the appended claims.

1. An ultrasonic handpiece comprising:
a body;
an ultrasonic generator movably provided on one side of the body;
a position adjustment unit provided on the body and configured to adjust a position of the ultrasonic generator so that a focusing position of ultrasonic waves of the ultrasonic generator is adjusted;
a position detection unit configured to detect a movement position of the ultrasonic generator; and
a control unit configured to control an output of the ultrasonic generator according to the position of the ultrasonic generator detected by the position detection unit.

2. The ultrasonic handpiece of claim 1, wherein:
the position adjustment unit includes
a dial rotatable relative to the body; and
an action rod provided inside the body, and having one side in contact with the dial to enable relative movement, and the other side connected to the ultrasonic generator and moved in an axial direction to correspond to a rotation of the dial.

3. The ultrasonic handpiece of claim 2, wherein:
a groove in a spiral shape is provided inside of the dial, and
a protrusion that moves along the groove is provided on the action rod.

4. The ultrasonic handpiece of claim 3, wherein:
the action rod has an axial direction period disposed in the axial direction and a radial direction period disposed in a radial direction with respect to the axial direction period,
the radial direction period has a bent end that is bent in the axial direction, and
the protrusion is provided on the bent end and elastically contacts the groove.

5. The ultrasonic handpiece of claim 4, wherein:
the protrusion has a curved shape that is convexly outward, and
a plurality of expansion portions expanded to correspond to a shape of the protrusion and spaced apart from each other are provided in the groove.

6. The ultrasonic handpiece of claim 3, wherein:
a pin is provided on one side of the protrusion, and
a pin guide portion is provided on one side of the groove to correspond to a movement trajectory of the pin.

7. The ultrasonic handpiece of claim 1, wherein:
the body includes
a main body; and
a cartridge detachably coupled to the main body,
the ultrasonic generator is provided inside the cartridge.

8. The ultrasonic handpiece of claim 7, wherein:
the action rod includes
a front rod connected to the ultrasonic generator;
a rear rod in contact with the dial; and
a middle rod having one end connected to the front rod and the other end connected to the rear rod.

9. The ultrasonic handpiece of claim 8, wherein:
the front rod and the middle rod respectively include magnetic coupling portions that are magnetically coupled to each other.

10. The ultrasonic handpiece of claim 8, wherein:
the middle rod and the rear rod include engaging coupling portions that are engaged and coupled to be constrained in an axial direction and a circumferential direction.

11. The ultrasonic handpiece of claim 7, wherein:
the position detection unit includes
a light emitting portion configured to irradiate light;
a light receiving portion configured to detect the light from the light emitting portion; and
a blocking portion configured to interoperate with an action rod and disposed to be movable in an axial direction between the light emitting portion and the light receiving portion to selectively block the light from the light emitting portion.

12. The ultrasonic handpiece of claim 11, wherein:
the light emitting portion includes a first light emitting portion and a second light emitting portion spaced apart in the axial direction,
the first light emitting portion is disposed closer to the ultrasonic generator than the second light emitting portion,
the blocking portion has an axial length capable of simultaneously blocking the first light emitting portion and the second light emitting portion, and
the dial and the action rod are configured to be fixed to a first position where the blocking portion blocks the second light emitting portion, a second position where the blocking portion simultaneously blocks the first light emitting portion and the second light emitting portion, and a third position where the blocking portion blocks the first light emitting portion.

13. The ultrasonic handpiece of claim 11, wherein:
the position detection unit includes
a first position detection unit including the light emitting portion and the blocking portion and provided inside the cartridge to detect a movement of the action rod; and
a second position detection unit including the light emitting portion and the blocking portion and provided inside the main body to detect the movement of the action rod.

14. The ultrasonic handpiece of claim 13, wherein:
the control unit controls driving of the ultrasonic generator to be stopped when detection results of the first position detection unit and the second position detection unit are different.

15. The ultrasonic handpiece of claim 14, further comprising:
a display unit provided on the body to display preset information on the outside of the body,
wherein the control unit controls the display unit to display an error on the outside of the body when the detection results of the first position detection unit and the second position detection unit are different.
